(19) United States
(12) Patent Application Publication
(10) Pub. No.: US 2026/0263740 A1
(43) Pub. Date: Sep. 10, 2026

(54) FEMALE URINARY CATHETER AND STRUCTURAL MEMBER THEREOF

(71) Applicant: WUHAN SILICON-BASED LIFE MEDICAL TECHNOLOGY CO., LTD, Wuhan (CN)

(72) Inventors: Qing LING, Wuhan (CN); Xiang ZHOU, Wuhan (CN); Pei WEI, Wuhan (CN)

(73) Assignee: WUHAN SILICON-BASED LIFE MEDICAL TECHNOLOGY CO., LTD, Wuhan (CN)

(21) Appl. No.: 19/660,333

(22) Filed: Apr. 28, 2026

Related U.S. Application Data
(63) Continuation of application No. PCT/CN2025/127968, filed on Oct. 16, 2025.

(30) Foreign Application Priority Data
Oct. 23, 2024 (CN) 202422558733.9
Sep. 8, 2025 (CN) 202521924254.2

(51) Int. Cl. A61M 25/00; A61M 25/01; A61M 25/04; A61M 39/24

(52) U.S. Cl. CPC A61M 25/0017; A61M 25/0043; A61M 25/0102; A61M 25/04; A61M 39/24; A61M 2039/248; A61M 2202/0014; A61M 2202/0496; A61M 2205/0216; A61M 2210/1092

(57) ABSTRACT

Provided in this disclosure is a female urinary catheter and a structural member thereof. The female urinary catheter includes a urinary catheter main body, a petal fixing member, a baffle fixing member, a catheter body sealing member, and a urinary catheterization valve. The urinary catheterization channel is formed in the urinary catheter main body in a penetrating manner, and an inner side wall of a catheter body of the urinary catheter main body is provided with a circle of the annular groove in a circumferential direction. The catheter body sealing member and the petal fixing member are both made of an elastic material. The catheter body sealing member is disposed on an outer side wall of the catheter body of the urinary catheter main body and corresponds to a position of the annular groove.

### CROSS-REFERENCE TO RELATED APPLICATIONS

The application claims priority to Chinese patent application No. 202422558733.9, filed on Oct. 23, 2024 and Chinese patent application No. 202521924254.2, filed on Sep. 8, 2025, the entire contents of which are incorporated herein by reference.

### TECHNICAL FIELD

This disclosure relates to the technical field of medical treatment, and in particular, to a female urinary catheter and a structural member thereof.

### BACKGROUND

Female urinary catheters are widely applied to clinical practice and are primarily applicable to patients with conditions such as urethral sphincter relaxation or neurological impairment of the urinary system, enabling them to achieve urinary control and urination functions through the devices. Existing urinary catheters usually require carrying a urine collection bag, which is inconvenient for patients in their daily lives. Furthermore, the unstable fit between the urinary catheter and the urethral wall easily leads to urine leakage. Due to the shorter physiological structure of the female urethra, the urinary catheter is difficult to secure and prone to slipping off.

Therefore, how to provide a new female urinary catheter and a structural member thereof for insertion into the female urethra to achieve controlled urination, with the technical effects of simple structure and convenient operation, is an urgent technical problem that those skilled in the art need to solve.

### SUMMARY

In view of the above problems, this disclosure aims to provide a female urinary catheter and a structural member thereof, to at least solve at least one of the above technical problems.

According to a first aspect, this disclosure provides a female urinary catheter, including:

a urinary catheter main body, where a urinary catheterization channel is formed in the urinary catheter main body in a penetrating manner, and an inner side wall of a catheter body of the urinary catheter main body is provided with a circle of an annular groove in a circumferential direction of the urinary catheter main body;

a petal fixing member, where the petal fixing member is fixed at an end portion of an insertion end of the urinary catheter main body in a surrounding manner in the circumferential direction of the urinary catheter main body;

a baffle fixing member, where the baffle fixing member is sleeved at an end portion of a urination end of the urinary catheter main body;

a catheter body sealing member, where the catheter body sealing member is disposed on an outer side wall of the catheter body of the urinary catheter main body and corresponds to a position of the annular groove; and

a urinary catheterization valve, where the urinary catheterization valve is fixedly disposed at the end portion of the urination end of the urinary catheter main body, the baffle fixing member is located between the urinary catheterization valve and the urinary catheter main body, and the urinary catheterization valve is used for controlling opening and closing of the urinary catheterization channel in the urinary catheter main body, where

the catheter body sealing member and the petal fixing member are both made of an elastic material.

Preferably, the baffle fixing member is of an elliptic structure, a fixing hole is formed in a central part of the elliptic structure in a penetrating manner, an inner diameter of the fixing hole is adapted to an outer diameter of the urinary catheter main body, and the elliptic structure is sleeved at a periphery of the end portion of the urination end of the urinary catheter main body through the fixing hole.

Preferably, the petal fixing member includes a plurality of fan-shaped blades, and the plurality of fan-shaped blades are arranged at the end portion of the insertion end of the urinary catheter main body at intervals in the circumferential direction of the urinary catheter main body.

Preferably, the catheter body sealing member is of an annular protrusion structure, an outer diameter of the annular protrusion structure is greater than the outer diameter of the urinary catheter main body, and a size of the annular protrusion structure is adapted to an expansion size of female urethra.

Preferably, an inner side wall of the insertion end of the urinary catheter main body is provided with a plurality of strip-shaped grooves of a preset length, and the strip-shaped grooves are formed by extending from the end portion of the insertion end to the urination end for the preset length.

Preferably, according to an aspect, the urinary catheterization valve includes:

an elastic valve body, where one end of the elastic valve body is fixedly connected to the end portion of the urination end of the urinary catheter main body, the elastic valve body is internally provided with a spherical hollow cavity, a side wall of the elastic valve body is provided with an inward hemispherical groove, the spherical hollow cavity is communicated with the hemispherical groove, the elastic valve body is further provided with an opening and closing channel communicated with the urinary catheterization channel in a penetrating manner in a length direction, and the opening and closing channel penetrates through the spherical hollow cavity;

a ball, where the ball is movably disposed in the spherical hollow cavity; and

an elastic sleeve, where the elastic sleeve is sleeved at a periphery of the elastic valve body, where

a size of the spherical hollow cavity is adapted to that of the ball, and an outer diameter of the opening and closing channel is less than an outer diameter of the spherical hollow cavity.

Preferably, the ball is made of a material conforming to biosecurity.

Preferably, inner side walls of two ends of the elastic sleeve are provided with a circle of a limiting protrusion respectively in a circumferential direction of the elastic sleeve, two ends of an outer side wall of the elastic valve body are provided with a circle of a limiting groove respectively in a circumferential direction, and the limiting protrusion is snapped into the limiting groove.

Preferably, the urinary catheter main body, the petal fixing member, the baffle fixing member, the catheter body sealing member, and the elastic valve body are integrally formed and are all made of an elastic material conforming to biosecurity.

Preferably, according to another aspect, the urinary catheterization valve includes:

a control valve body, where one end of the control valve body is fixedly connected to the end portion of the urination end of the urinary catheter main body, the control valve body is internally provided with a spherical groove, a side wall of the control valve body extends outward to form a spherical cavity, the spherical cavity is communicated with the spherical groove, the control valve body is further provided with a urination channel communicated with the urinary catheterization channel in a penetrating manner in a length direction, and the urination channel penetrates through the spherical hollow cavity; and

a moving ball, where the moving ball is movably disposed in the spherical groove, where

a size of the spherical groove is adapted to that of the moving ball, and an outer diameter of the urination channel is less than an outer diameter of the spherical groove.

According to a second aspect, this disclosure further provides a female urinary catheter, including:

a urinary catheter main body, where the urinary catheter main body is provided with a urinary catheterization channel in a penetrating manner in a length direction;

an umbrella-shaped anti-off member, where a top of the umbrella-shaped anti-off member is provided with a guide wire abutting end, the umbrella-shaped anti-off member is internally provided with a guide wire abutting hollow cavity, an opening end of the guide wire abutting hollow cavity is fixedly connected to an end portion of an insertion end of the urinary catheter main body, and a side wall of the umbrella-shaped anti-off member is provided with a urine inlet;

a baffle fixing member, where the baffle fixing member is sleeved at a periphery of a urination end of the urinary catheter main body;

a urinary catheterization valve, where the urinary catheterization valve is fixedly disposed at an end portion of the urination end of the urinary catheter main body, and the baffle fixing member is located between the urinary catheterization valve and the urinary catheter main body; and

a traction guide wire, where the traction guide wire and the urinary catheter main body are disposed independently, and one end of the traction guide wire abuts against the guide wire abutting end after sequentially passing through the urinary catheterization channel and the guide wire abutting hollow cavity, where

the umbrella-shaped anti-off member is made of an elastic material, and the guide wire abutting hollow cavity, the urinary catheterization channel, and the urine inlet are communicated with each other; and an outer diameter of the umbrella-shaped anti-off member in a natural state is greater than an outer diameter of the urinary catheter main body, and an outer diameter of the umbrella-shaped anti-off member in a deformed state is equivalent to the outer diameter of the urinary catheter main body.

Preferably, the baffle fixing member is of an elliptic structure, a fixing hole is formed in a central part of the elliptic structure in a penetrating manner, an inner diameter of the fixing hole is adapted to the outer diameter of the urinary catheter main body, and the elliptic structure is sleeved at a periphery of the end portion of the urination end of the urinary catheter main body through the fixing hole; and

an inner side wall of a catheter body of the urinary catheter main body is provided with a circle of an annular groove in a circumferential direction of the urinary catheter main body, an outer side wall of the catheter body of the urinary catheter main body is provided with a catheter body sealing member, the catheter body sealing member corresponds to a position of the annular groove, the catheter body sealing member is of an annular protrusion structure, an outer diameter of the annular protrusion structure is greater than the outer diameter of the urinary catheter main body, and a size of the annular protrusion structure is adapted to an expansion size of female urethra.

Preferably, the urinary catheterization valve includes:

a control valve body, where one end of the control valve body is fixedly connected to the end portion of the urination end of the urinary catheter main body, the control valve body is internally provided with a spherical groove, a side wall of the control valve body extends outward to form a spherical cavity, the spherical cavity is communicated with the spherical groove, the control valve body is provided with a urination channel communicated with the urinary catheterization channel in a penetrating manner in a length direction, and the urination channel penetrates through the spherical groove; and

a moving ball, where the moving ball is movably disposed in the spherical groove, where

the spherical groove is in interference abutting with the moving ball, and an outer diameter of the urination channel is less than an outer diameter of the spherical groove.

Preferably, the urinary catheter main body, the umbrella-shaped anti-off member, the baffle fixing member, the catheter body sealing member, and the control valve body are integrally formed and are all made of an elastic material conforming to biosecurity.

According to a third aspect, this disclosure further provides a structural member for a female urinary catheter, where the female urinary catheter includes a urinary catheter main body, and the urinary catheter main body is provided with a urinary catheterization channel; the structural member includes: a urinary catheterization valve, where the urinary catheterization valve is disposed at a urination end of the urinary catheter main body; and the structural member further includes:

a petal fixing member, where the petal fixing member is disposed at an insertion end of the urinary catheter main body in a surrounding manner in a circumferential direction of the urinary catheter main body;

or

an umbrella-shaped anti-off member, where the umbrella-shaped anti-off member is provided with a guide wire abutting end, the umbrella-shaped anti-off member is internally provided with a guide wire abutting hollow cavity, an opening end of the guide wire abutting hollow cavity is fixedly connected to the insertion end of the urinary catheter main body, the umbrella-shaped anti-off member is provided with a urine inlet, and the guide wire abutting hollow cavity, the urinary catheterization channel, and the urine inlet are communicated with each other.

Compared with the prior art, the beneficial effects of this disclosure are as follows:

Specifically, this disclosure provides a female urinary catheter, including the urinary catheter main body, the petal fixing member, the baffle fixing member, the catheter body sealing member, and the urinary catheterization valve. The urinary catheterization channel is formed in the urinary catheter main body in a penetrating manner. The inner side wall of the catheter body of the urinary catheter main body is provided with a circle of the annular groove in the circumferential direction of the urinary catheter main body. The catheter body sealing member and the petal fixing member are both made of the elastic material. The catheter body sealing member is disposed on the outer side wall of the catheter body of the urinary catheter main body and corresponds to the position of the annular groove, so that the catheter body sealing member is attached to a urethral wall for sealing. At the same time, by forming the annular groove, the catheter body sealing member has a compression space, so that a persistent injury caused by the catheter body sealing member to the urethral wall may be avoided. The petal fixing member is fixed at the end portion of the insertion end of the urinary catheter main body in a surrounding manner in the circumferential direction of the urinary catheter main body, to achieve engagement with a bladder orifice of a patient through the petal fixing member, thereby preventing the urinary catheter from slipping out. The baffle fixing member is sleeved at the end portion of the urination end of the urinary catheter main body, to achieve engagement at a urethral orifice through the baffle fixing member, thereby preventing the urinary catheter from contracting inward and slipping into a bladder. The urinary catheterization valve is fixedly disposed at the end portion of the urination end of the urinary catheter main body. The baffle fixing member is located between the urinary catheterization valve and the urinary catheter main body. The urinary catheterization valve is retained outside the patient and may be concealed in female labia, to manually operate the urinary catheterization valve to control the opening and closing of the urinary catheterization channel in the urinary catheter main body. This disclosure is simple in structure and convenient in operation.

The above description is only an overview of the technical solutions of this disclosure. To understand the technical means of this disclosure more clearly to implement them in accordance with the content of the specification, and to make the above and other objectives, features, and advantages of this disclosure more obvious and understandable, the specific implementations of this disclosure are illustrated below.

### BRIEF DESCRIPTION OF THE DRAWINGS

To illustrate the technical solutions in the embodiments of this disclosure or in the prior art more clearly, the following briefly introduces the accompanying drawings to be used for describing the embodiments. Obviously, the accompanying drawings in the following description only show some embodiments of this disclosure, and those of ordinary skill in the art may still derive other accompanying drawings from these accompanying drawings without creative efforts.

FIG. 1 is a schematic structural diagram of a female urinary catheter during urination according to Embodiment 1 of this disclosure;

FIG. 2 is a schematic structural diagram of a female urinary catheter during urinary control according to Embodiment 1 of this disclosure;

FIG. 3 is a schematic three-dimensional structural diagram of a female urinary catheter according to Embodiment 1 of this disclosure;

FIG. 4 is a schematic structural diagram of another urinary catheterization valve during urination according to Embodiment 1 of this disclosure;

FIG. 5 is a schematic structural diagram of another urinary catheterization valve during urinary control according to Embodiment 1 of this disclosure;

FIG. 6 is a schematic structural diagram of a combination of a female urinary catheter and a guide wire according to Embodiment 2 of this disclosure;

FIG. 7 is a schematic diagram of deformation of a female urinary catheter during delivery according to Embodiment 2 of this disclosure;

FIG. 8 is a schematic structural diagram of a female urinary catheter according to Embodiment 2 of this disclosure;

FIG. 9 is a sectional view of a female urinary catheter according to Embodiment 2 of this disclosure; and

FIG. 10 is a schematic structural diagram of a traction guide wire according to Embodiment 2 of this disclosure.

### REFERENCE NUMERALS

1. urinary catheter main body;
11. urinary catheterization channel;
12. annular groove;
13. strip-shaped groove;
2. petal fixing member;
21. fan-shaped blade;
3. baffle fixing member;
4. catheter body sealing member;
5. urinary catheterization valve;
51. elastic valve body;
511. spherical hollow cavity;
512. hemispherical groove;
513. opening and closing channel;
514. limiting groove;
52. ball;
53. elastic sleeve;
531. limiting protrusion;
61. control valve body;
611. spherical groove;
612. spherical cavity;
613. urination channel;
62. moving ball;
7. umbrella-shaped anti-off member;
71. guide wire abutting end;
72. guide wire abutting hollow cavity;
73. urine inlet;
8. traction guide wire.

### DETAILED DESCRIPTION OF THE EMBODIMENTS

The technical solutions in the embodiments of this disclosure are clearly and completely described below with reference to the accompanying drawings in the embodiments of this disclosure. Obviously, the described embodiments are only some but not all of the embodiments of this disclosure. All other embodiments obtained by those skilled in the art based on the embodiments of the specification shall fall within the protection scope of this disclosure. The term “and/or” involved in the embodiments represents both situations of “and” and “or”. In other words, A and/or B mentioned in the embodiments of the specification represent two situations: A and B, and A or B, and describe three states in which A and B exist, for example, A and/or B represent that only A rather than B is included; only B rather than A is included; and both A and B are included.

Also, in the embodiments of the specification, when an assembly is said to be “connected” to another assembly, it may be directly connected to the another assembly, or there may be an intervening assembly at the same time. When an assembly is said to be “disposed” on another assembly, it may be directly disposed on the another assembly, or there may be an intervening assembly at the same time.

The following illustrates the implementations of this disclosure through specific examples, and those skilled in the art can easily understand other advantages and beneficial effects of this disclosure from the content disclosed in the specification. This disclosure can also be implemented or applied in other different implementations, and various details in the specification can also be modified or changed in various ways in accordance with different ideas and applications without departing from the spirit of this disclosure.

### Embodiment 1

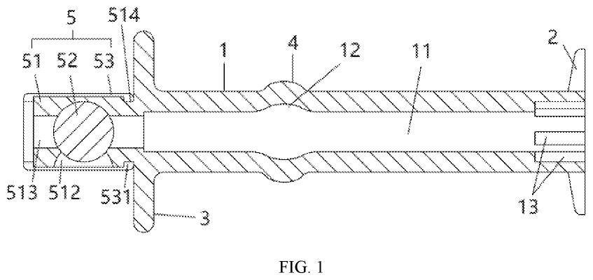

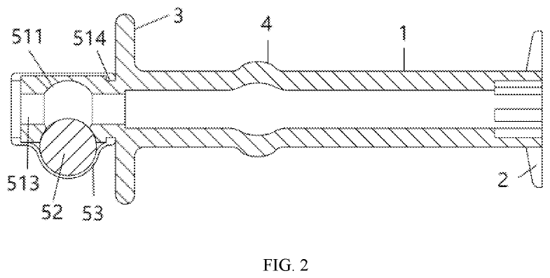

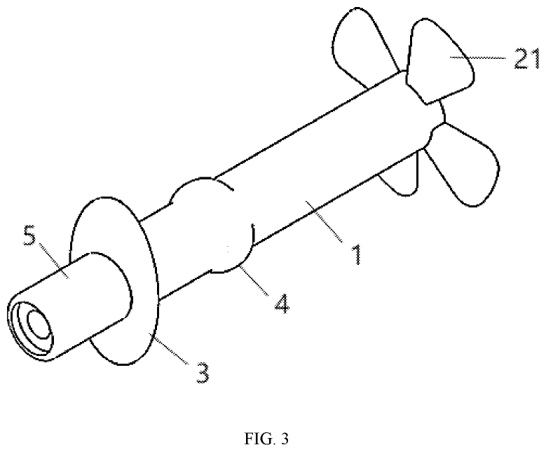

Referring to FIG. 1 to FIG. 3, specifically, in an implementation of this female urinary catheter, a female urinary catheter provided by this disclosure is used for being placed into female urethra to achieve controlled urination. The female urinary catheter specifically includes a urinary catheter main body 1, a petal fixing member 2, a baffle fixing member 3, a catheter body sealing member 4, and a urinary catheterization valve 5. A urinary catheterization channel 11 is formed in the urinary catheter main body 1 in a penetrating manner, and an inner side wall of a catheter body of the urinary catheter main body 1 is provided with a circle of an annular groove 12 in a circumferential direction of the urinary catheter main body 1. The petal fixing member 2 is fixed at an end portion of an insertion end of the urinary catheter main body 1 in a surrounding manner in the circumferential direction of the urinary catheter main body 1. The baffle fixing member 3 is sleeved at an end portion of a urination end of the urinary catheter main body 1. The catheter body sealing member 4 is disposed on an outer side wall of the catheter body of the urinary catheter main body 1 and corresponds to the position of the annular groove 12. The urinary catheterization valve 5 is fixedly disposed at the end portion of the urination end of the urinary catheter main body 1, the baffle fixing member 3 is located between the urinary catheterization valve 5 and the urinary catheter main body 1, and the urinary catheterization valve 5 is used for controlling the opening and closing of the urinary catheterization channel 11 in the urinary catheter main body 1. The catheter body sealing member 4 and the petal fixing member 2 are both made of an elastic material.

Specifically, an embodiment of this disclosure provides a female urinary catheter, including the urinary catheter main body 1, the petal fixing member 2, the baffle fixing member 3, the catheter body sealing member 4, and the urinary catheterization valve 5. The urinary catheterization channel 11 is formed in the urinary catheter main body 1 in a penetrating manner. The inner side wall of the catheter body of the urinary catheter main body 1 is provided with a circle of the annular groove 12 in the circumferential direction of the urinary catheter main body 1. The catheter body sealing member 4 and the petal fixing member 2 are both made of the elastic material, allowing them to be folded under external force to facilitate placement into the female urethra. The catheter body sealing member 4 is disposed on the outer side wall of the catheter body of the urinary catheter main body 1 and corresponds to the position of the annular groove 12, so that the catheter body sealing member 4 is attached to a urethral wall for sealing. At the same time, by forming the annular groove 12, the catheter body sealing member 4 has a compression space, so that a persistent injury caused by the catheter body sealing member 4 to the urethral wall may be avoided. The petal fixing member 2 is fixed at the end portion of the insertion end of the urinary catheter main body 1 in a surrounding manner in the circumferential direction of the urinary catheter main body 1, to achieve engagement with a bladder orifice of a patient through the petal fixing member 2, thereby preventing the urinary catheter from slipping out. The baffle fixing member 3 is sleeved at the end portion of the urination end of the urinary catheter main body 1, to achieve engagement at a urethral orifice through the baffle fixing member 3, thereby preventing the urinary catheter from contracting inward and slipping into a bladder. The urinary catheterization valve 5 is fixedly disposed at the end portion of the urination end of the urinary catheter main body 1. The baffle fixing member 3 is located between the urinary catheterization valve 5 and the urinary catheter main body 1. The urinary catheterization valve 5 is retained outside the patient and may be concealed in female labia, to manually operate the urinary catheterization valve 5 to control the opening and closing of the urinary catheterization channel 11 in the urinary catheter main body 1. This disclosure is simple in structure and convenient in operation.

The female urinary catheter of this disclosure may be compressed and loaded into a hand-tear sheath. Therefore, the catheter body sealing member 4 and the petal fixing member 2 of this disclosure are both required to be disposed as elastic structures, facilitating compression and loading into the hand-tear sheath. When in use, the hand-tear sheath is delivered to the bladder orifice via the urethral orifice, and a pushing rod is used to push the urinary catheter. At the same time, the hand-tear sheath is torn open and retracted; the petal fixing member 2 is released at the bladder orifice, and the position is appropriately adjusted; and the sheath continues to be retracted until the baffle is anchored at the urethral orifice. The position of the urinary catheter is fixed through the petal fixing member 2 and the baffle fixing member 3.

In a possible implementation, the baffle fixing member 3 may be disposed in an elliptic structure. A fixing hole is formed at a central part of the elliptic structure in a penetrating manner. The inner diameter of the fixing hole is adapted to the outer diameter of the urinary catheter main body 1. The elliptic structure is sleeved at the periphery of the end portion of the urination end of the urinary catheter main body 1 through the fixing hole, to achieve engagement at the urethral orifice through the elliptic structure, thereby preventing the urinary catheter from contracting inward and slipping into the bladder. Disposing in the elliptic structure may reduce the widths of both left and right sides of the baffle fixing member 3, facilitating concealment of the baffle fixing member 3 in the female labia, so that the squeezing of both the left and right sides on the labia may be reduced. At the same time, a narrow and elongated shape of the elliptic structure is more in line with a physiological structure of female external genitalia, which can be covered by tissue to reduce foreign body sensation and can also be positioned axially without rotating at any angle.

In a possible implementation, the petal fixing member 2 includes a plurality of fan-shaped blades 21. The plurality of fan-shaped blades 21 are arranged at the end portion of the insertion end of the urinary catheter main body 1 at intervals in the circumferential direction of the urinary catheter main body 1.

Further, an inner side wall of the insertion end of the urinary catheter main body 1 is provided with a plurality of strip-shaped grooves 13 of a preset length. The strip-shaped grooves 13 are formed by extending from the end portion of the insertion end to the urination end for the preset length.

Specifically, the petal fixing member 2 includes the plurality of fan-shaped blades 21, and the plurality of fan-shaped blades 21 are arranged at intervals, so that there is a gap between two adjacent fan-shaped blades 21, facilitating folding of the fan-shaped blades under external force. The petal fixing member 2 is in a contracted state under external force with the diameter in the contracted state less than the diameter of the urethra, and is in an expanded state in a natural state with the diameter in the expanded state greater than the diameters of the urethra and the bladder orifice. The inner side wall of the insertion end of the urinary catheter main body 1 is provided with the plurality of strip-shaped grooves 13 of the preset length, so that contraction of the petal fixing member 2 is facilitated. The preset length is far less than the length of the urinary catheter main body 1. By forming the strip-shaped grooves, during delivery, there is a sufficient deformation space for the petal fixing member to be folded and superimposed, and the connection strength between the petal and the main body may be adjusted through the widths of the grooves, thereby adjusting the fixing strength of the petal in the body.

In a possible implementation, the catheter body sealing member 4 is of an annular protrusion structure. The outer diameter of the annular protrusion structure is greater than the outer diameter of the urinary catheter main body 1. The size of the annular protrusion structure is adapted to the expansion size of the female urethra, to achieve tight attachment with the urethral wall through the annular protrusion structure, thereby playing a sealing role and preventing urine leakage.

In a possible implementation, the urinary catheterization valve 5 includes an elastic valve body 51, a ball 52, and an elastic sleeve 53. One end of the elastic valve body 51 is fixedly connected to the end portion of the urination end of the urinary catheter main body 1. The elastic valve body 51 is internally provided with a spherical hollow cavity 511, a side wall of the elastic valve body 51 is provided with an inward hemispherical groove 512, and the spherical hollow cavity 511 is communicated with the hemispherical groove 512. The elastic valve body 51 is further provided with an opening and closing channel 513 communicated with the urinary catheterization channel 11 in a penetrating manner in a length direction, and the opening and closing channel 513 penetrates through the spherical hollow cavity 511. The ball 52 is movably disposed in the spherical hollow cavity 511. The elastic sleeve 53 is sleeved at the periphery of the elastic valve body 51. The size of the spherical hollow cavity 511 is adapted to that of the ball 52, and the outer diameter of the opening and closing channel 513 is less than the outer diameter of the spherical hollow cavity 511.

Specifically, when a squeezing force is applied to two sides of the elastic valve body 51 or an opposite side of the hemispherical groove 512, the ball 52 enters the hemispherical groove 512 from the spherical hollow cavity 511 and squeezes and jacks up the elastic sleeve 53. At the same time, the opening and closing channel 513 in the elastic valve body 51 is opened, achieving a urination function. When urination is completed, a protrusion position of the elastic sleeve 53 is squeezed to press the ball 52 back to the spherical hollow cavity 511 in the elastic valve body 51. The ball 52 is attached to a side wall of the spherical hollow cavity 511 to block and close the middle opening and closing channel 513, achieving a urinary control effect.

In a possible implementation, inner side walls of two ends of the elastic sleeve 53 are provided with a circle of a limiting protrusion 531 respectively in a circumferential direction of the elastic sleeve 53. Two ends of an outer side wall of the elastic valve body 51 are provided with a circle of a limiting groove 514 respectively in a circumferential direction. The limiting protrusion 531 is snapped into the limiting groove 514. The limiting protrusion 531 is snapped into the limiting groove 514, so that the elastic sleeve 53 is limited, thereby avoiding displacement or detachment of the elastic sleeve 53 when the ball 52 is squeezed into the hemispherical groove 512 from the spherical hollow cavity 511 to enable the elastic sleeve 53 to deform.

In a possible implementation, the surface of the ball 52 is smooth, and the surface thereof may be made of a material conforming to biosecurity, for example, polytetrafluoroethylene, quartz glass, polyethylene, or stainless steel. A main body of the ball may be made of an elastic material such as silicone, latex rubber, TPU, or TPE, to reduce the weight of the ball 52 while ensuring safety, thereby improving usage comfort.

In a possible implementation, the urinary catheter main body 1, the petal fixing member 2, the baffle fixing member 3, the catheter body sealing member 4, and the elastic valve body 51 are integrally formed and are all made of an elastic material conforming to biosecurity, which specifically may be made of silicone, to improve usage comfort while simplifying the overall structure.

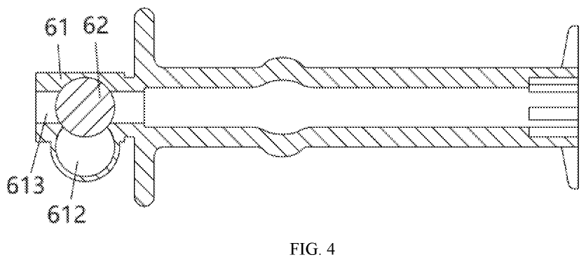

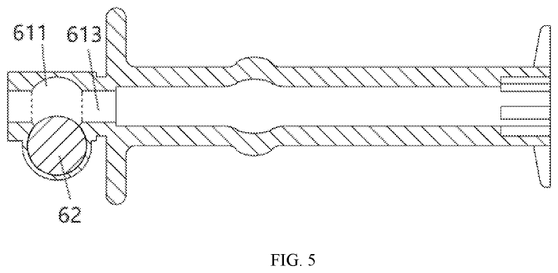

As a replaceable implementation, this disclosure provides a structural design of another urinary catheterization valve. As shown in FIG. 4 and FIG. 5, the urinary catheterization valve includes a control valve body 61 and a moving ball 62. One end of the control valve body 61 is fixedly connected to the end portion of the urination end of the urinary catheter main body 1. The control valve body 61 is internally provided with a spherical groove 611, a side wall of the control valve body 61 extends outward to form a spherical cavity 612, and the spherical cavity 612 is communicated with the spherical groove 611. The control valve body 61 is further provided with a urination channel 613 communicated with the urinary catheterization channel 11 in a penetrating manner in the length direction, and the urination channel 613 penetrates through the spherical hollow cavity 511. The moving ball 62 is movably disposed in the spherical groove 611. The size of the spherical groove 611 is adapted to that of the moving ball 62, and the outer diameter of the urination channel 613 is less than the outer diameter of the spherical groove 611.

Specifically, when a squeezing force is applied to an opposite side of the spherical cavity 612 of the control valve body 61, the moving ball 62 enters the spherical cavity 612 from the spherical groove 611, so that the urination channel 613 in the control valve body 61 is opened, achieving the urination function, and a specific structural example is shown in FIG. 5. When urination is completed, the moving ball 62 at the spherical cavity 612 is squeezed to press the moving ball 62 back to the spherical groove 611 in the control valve body 61. The moving ball 62 is attached to a side wall of the spherical groove 611 to block and close the middle urination channel 613, achieving the urinary control effect, and a specific structural example is shown in FIG. 4.

The urinary catheter main body 1, the petal fixing member 2, the baffle fixing member 3, the catheter body sealing member 4, and the control valve body 61 are integrally formed and are all made of an elastic material conforming to biosecurity, which specifically may be silicone, to improve usage comfort while simplifying the overall structure.

### Embodiment 2

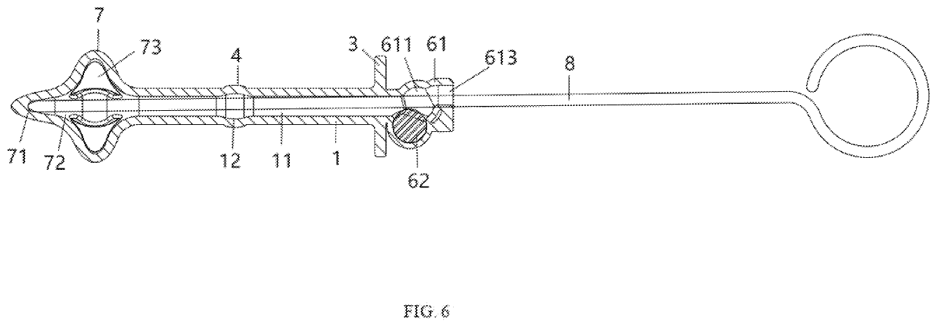

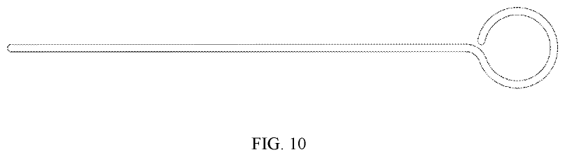

This embodiment further provides a female urinary catheter. As shown in FIG. 6 to FIG. 10, the female urinary catheter provided in this embodiment includes: a urinary catheter main body 1, an umbrella-shaped anti-off member 7, a baffle fixing member 3, a urinary catheterization valve, and a traction guide wire 8. The urinary catheter main body 1 is provided with a urinary catheterization channel 11 in a penetrating manner in a length direction. The top of the umbrella-shaped anti-off member 7 is provided with a guide wire abutting end 71. The umbrella-shaped anti-off member 7 is internally provided with a guide wire abutting hollow cavity 72. An opening end of the guide wire abutting hollow cavity 72 is fixedly connected to an end portion of an insertion end of the urinary catheter main body 1. A side wall of the umbrella-shaped anti-off member 7 is provided with a urine inlet 73. A plurality of the urine inlets 73 may be uniformly formed at intervals in a circumferential direction of the umbrella-shaped anti-off member 7. The baffle fixing member 3 is sleeved at the periphery of a urination end of the urinary catheter main body 1. The urinary catheterization valve is fixedly disposed at an end portion of the urination end of the urinary catheter main body 1. The baffle fixing member 3 is located between the urinary catheterization valve and the urinary catheter main body 1. The traction guide wire 8 and the urinary catheter main body 1 are disposed independently. One end of the traction guide wire 8 abuts against the guide wire abutting end 71 after sequentially passing through the urinary catheterization channel 11 and the guide wire abutting hollow cavity 72. The umbrella-shaped anti-off member 7 is made of an elastic material, and the guide wire abutting hollow cavity 72, the urinary catheterization channel 11, and the urine inlet 73 are communicated with each other. The outer diameter of the umbrella-shaped anti-off member 7 in a natural state is greater than the outer diameter of the urinary catheter main body 1, and the outer diameter of the umbrella-shaped anti-off member 7 in a deformed state is equivalent to the outer diameter of the urinary catheter main body 1.

Specifically, through the structural matching between the traction guide wire 8 and the umbrella-shaped anti-off member 7, the urinary catheter main body 1 is delivered into urethra of a patient from a urethral orifice of the patient through the traction guide wire 8. Specifically, one end of the traction guide wire 8 abuts against the guide wire abutting end 71 after sequentially passing through the urinary catheterization channel 11 and the guide wire abutting hollow cavity 72. Under the abutting effect of the traction guide wire 8, the umbrella-shaped anti-off member 7 deforms axially, so that the outer diameter of the deformed umbrella-shaped anti-off member 7 is equivalent to the outer diameter of the urinary catheter main body 1, thereby directly pushing a part of the umbrella-shaped anti-off member 7 and the urinary catheter main body 1 into the urethra of the female patient through the traction guide wire 8 and releasing and taking out the traction guide wire 8 when the umbrella-shaped anti-off member 7 reaches a bladder orifice. At this time, the umbrella-shaped anti-off member 7 recovers from deformation and engages with the bladder orifice to limit the urinary catheter main body 1. The baffle fixing member 3 and the urinary catheterization valve are located outside the urethral orifice of the patient, that is, located outside the patient. Two ends of the urinary catheter main body 1 are limited through the umbrella-shaped anti-off member 7 and the baffle fixing member 3, to prevent the urinary catheter from slipping off. The urinary catheterization valve is located outside the patient, so that it is convenient for the patient to manually operate the opening and closing of the urinary catheterization valve to control the opening and closing of the urinary catheterization channel 11 in the urinary catheter main body 1. This disclosure is simple in structure and convenient in operation.

The length of the traction guide wire 8 is greater than the length of the deformed urinary catheter. One end of the traction guide wire 8 is provided with a grip portion, to facilitate manual grip operations. The traction guide wire 8 may be made of a material such as a nickel-titanium alloy or medical-grade stainless steel, to meet the requirements of biocompatibility and mechanical strength. An umbrella-shaped fixing member is stretched through the traction guide wire, so that the overall umbrella-shaped fixing member may be delivered to a target position via a natural channel after deformation and contraction.

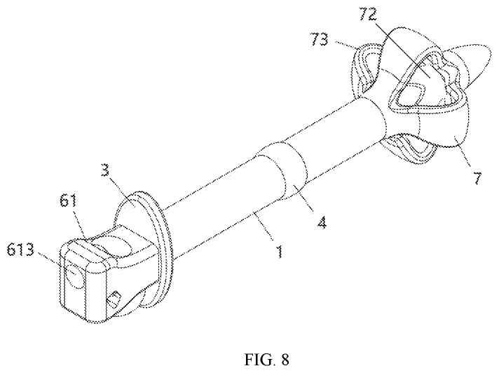

In a possible implementation, as shown in FIG. 8, the umbrella-shaped anti-off member 7 is formed by a plurality of arc-shaped umbrella segments that are connected in a surrounding manner in a circumferential direction. A surrounded region among the plurality of arc-shaped umbrella segments forms the guide wire abutting hollow cavity 72. Ends of the plurality of arc-shaped umbrella segments are connected to form the guide wire abutting end 71, and the other ends of the plurality of arc-shaped umbrella segments are connected to form the opening end of the guide wire abutting hollow cavity 72. The urine inlet 73 is formed between every two arc-shaped umbrella segments, and the urine inlet 73 and the guide wire abutting hollow cavity 72. By setting the size of an opening of the urine inlet 73, the fixing strength of the umbrella-shaped anti-off member in the body is adjusted. Specifically, the size may be correspondingly set according to actual requirements.

In a possible implementation, the baffle fixing member 3 is of an elliptic structure. A fixing hole is formed at a central part of the elliptic structure in a penetrating manner. The inner diameter of the fixing hole is adapted to the outer diameter of the urinary catheter main body 1. The elliptic structure is sleeved at the periphery of the end portion of the urination end of the urinary catheter main body 1 through the fixing hole, to achieve engagement at the urethral orifice through the elliptic structure, thereby preventing the urinary catheter from contracting inward and slipping into the bladder. At the same time, disposing in the elliptic structure reduces the widths of both left and right sides of the baffle fixing member 3, facilitating concealment of the baffle fixing member 3 in labia, so that the squeezing of both the left and right sides on the labia may be reduced.

In a possible implementation, an inner side wall of a catheter body of the urinary catheter main body 1 is provided with a circle of an annular groove 12 in a circumferential direction of the urinary catheter main body 1. An outer side wall of the catheter body of the urinary catheter main body 1 is provided with a catheter body sealing member 4. The catheter body sealing member 4 corresponds to the position of the annular groove 12. The catheter body sealing member 4 is of an annular protrusion structure. The outer diameter of the annular protrusion structure is greater than the outer diameter of the urinary catheter main body 1. The size of the annular protrusion structure is adapted to the expansion size of the female urethra.

Specifically, the catheter body sealing member 4 is disposed on the outer side wall of the catheter body of the urinary catheter main body 1 and corresponds to the position of the annular groove 12, so that the catheter body sealing member 4 is attached to a urethral wall for sealing. At the same time, by forming the annular groove 12, the catheter body sealing member 4 has a compression space, so that a persistent injury caused by the catheter body sealing member 4 to the urethral wall may be avoided. The catheter body sealing member 4 is of the annular protrusion structure. The outer diameter of the annular protrusion structure is greater than the outer diameter of the urinary catheter main body 1. The size of the annular protrusion structure is adapted to the expansion size of the female urethra, to achieve tight attachment with the urethral wall through the annular protrusion structure, thereby playing a sealing role and preventing urine leakage.

In a possible implementation, the urinary catheterization valve includes a control valve body 61 and a moving ball 62. One end of the control valve body 61 is fixedly connected to the end portion of the urination end of the urinary catheter main body 1. The control valve body 61 is internally provided with a spherical groove 611, a side wall of the control valve body 61 extends outward to form a spherical cavity 612, and the spherical cavity 612 is communicated with the spherical groove 611. The control valve body 61 is further provided with a urination channel 613 communicated with the urinary catheterization channel 11 in a penetrating manner in the length direction, and the urination channel 613 penetrates through the spherical groove 611. The moving ball 62 is movably disposed in the spherical groove 611. The spherical groove 611 is in interference abutting with the moving ball 62, and the outer diameter of the urination channel 613 is less than the outer diameter of the spherical groove 611.

Further, the urinary catheter main body 1, the umbrella-shaped anti-off member 7, the baffle fixing member 3, the catheter body sealing member 4, and the control valve body 61 are integrally formed and are all made of an elastic material conforming to biosecurity, which specifically may be silicone, to improve usage comfort while simplifying the overall structure.

Specifically, when a squeezing force is applied to an opposite side of the spherical cavity 612 of the control valve body 61, the moving ball 62 enters the spherical cavity 612 from the spherical groove 611, so that the urination channel 613 in the control valve body 61 is opened, achieving the urination function, and a specific structural example is shown in FIG. 6 to FIG. 9. When urination is completed, the moving ball 62 at the spherical cavity 612 is squeezed to press the moving ball 62 back to the spherical groove 611 in the control valve body 61. The moving ball 62 is attached to a side wall of the spherical groove 611 to block and close the middle urination channel 613, achieving the urinary control effect.

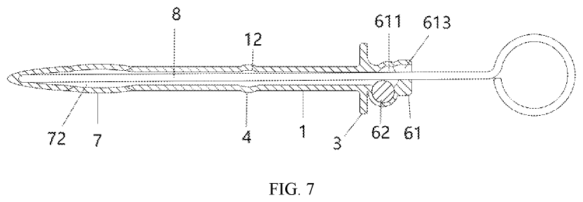

When in specific use, in the natural state, the traction guide wire 8 first extends into the guide wire abutting hollow cavity 72 from the urination channel 613 and the urinary catheterization channel 11 sequentially and abuts against the guide wire abutting end 71 to extend and deform the umbrella-shaped anti-off member 7. Specifically, as shown in FIG. 7, the outer diameter of the deformed umbrella-shaped anti-off member 7 gradually decreases until it is finally the same as the outer diameter of the urinary catheter main body 1. At this time, one end of the umbrella-shaped anti-off member 7 is first delivered to the bladder orifice from the urethral orifice of the female patient, and the umbrella-shaped anti-off member 7 is released to enable the umbrella-shaped anti-off member 7 to recover to the natural state and engage with the bladder orifice. At this time, the baffle fixing member 3 engages with the urethral orifice of the patient, thereby anchoring the position of the urinary catheter of this disclosure. At this time, the urinary catheterization valve of the urinary catheter is located outside the urethral orifice of the patient, and the patient may control urination by squeezing the urinary catheterization valve.

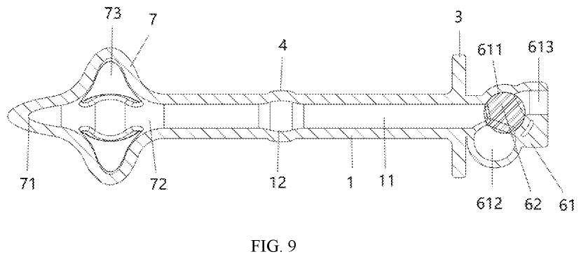

In a possible implementation, by using an axial direction of the urinary catheter main body 1 as a reference, there is a preset deflection angle between a straight line connecting a sphere center of the spherical cavity 612 and a sphere center of the spherical groove 611 and the axial direction of the urinary catheter main body 1, which is specifically shown in FIG. 6, FIG. 7, and FIG. 9. The spherical cavity 612 and the spherical groove 611 may partially overlap as long as the urinary control and urination functions are not affected. The accommodation diameter of the spherical cavity 612 may be slightly greater than the diameter of the moving ball 62, and the accommodation diameter of the spherical groove 611 may be slightly less than the diameter of the moving ball 62, to achieve an interference fit with the moving ball 62, thereby improving the stability of urinary control.

### Embodiment 3

This embodiment further provides a structural member for a female urinary catheter. The female urinary catheter includes a urinary catheter main body, and the urinary catheter main body is provided with a urinary catheterization channel. The structural member includes: a urinary catheterization valve, where the urinary catheterization valve is disposed at a urination end of the urinary catheter main body. The structural member further includes: a petal fixing member, where the petal fixing member is disposed at an insertion end of the urinary catheter main body in a surrounding manner in a circumferential direction of the urinary catheter main body; or an umbrella-shaped anti-off member, where the umbrella-shaped anti-off member is provided with a guide wire abutting end, the umbrella-shaped anti-off member is internally provided with a guide wire abutting hollow cavity, an opening end of the guide wire abutting hollow cavity is connected to the insertion end of the urinary catheter main body, the umbrella-shaped anti-off member is provided with a urine inlet, and the guide wire abutting hollow cavity, the urinary catheterization channel, and the urine inlet are communicated with each other.

Specifically, one end of the urinary catheter main body is the insertion end, and the other end of the urinary catheter main body is the urination end. By fixedly disposing the urinary catheterization valve for urinary control and urination at the urination end, after the urinary catheter is internally disposed, the urinary catheterization valve is retained at a urethral orifice of a patient, so that it is convenient for the patient to manually operate the opening and closing of the urinary catheterization valve to control the opening and closing of the urinary catheterization channel in the urinary catheter main body to achieve urination and urinary control. By disposing the petal fixing member or the umbrella-shaped anti-off member at the insertion end of the urinary catheter main body, to achieve engagement at a bladder orifice of the patient through the petal fixing member or the umbrella-shaped anti-off member. The petal fixing member is fixed at an end portion of the insertion end of the urinary catheter main body in a surrounding manner in the circumferential direction of the urinary catheter main body. The top of the umbrella-shaped anti-off member is provided with the guide wire abutting end. The umbrella-shaped anti-off member is internally provided with the guide wire abutting hollow cavity. The opening end of the guide wire abutting hollow cavity is fixedly connected to the end portion of the insertion end of the urinary catheter main body. A side wall of the umbrella-shaped anti-off member is provided with the urine inlet. Specifically, a plurality of the urine inlets may be uniformly disposed at intervals in the circumferential direction. The guide wire abutting hollow cavity, the urinary catheterization channel, and the urine inlet are communicated with each other. The umbrella-shaped anti-off member is made of an elastic material. The outer diameter of the umbrella-shaped anti-off member in a natural state is greater than the outer diameter of the urinary catheter main body, and the outer diameter of the umbrella-shaped anti-off member in a deformed state is equivalent to the outer diameter of the urinary catheter main body.

The outer diameter of the umbrella-shaped anti-off member in the natural state is greater than the outer diameter of the urinary catheter main body, and the outer diameter of the umbrella-shaped anti-off member in the deformed state is equivalent to the outer diameter of the urinary catheter main body.

In this embodiment, an inner side wall of a catheter body of the urinary catheter main body is provided with a circle of an annular groove in the circumferential direction, and an outer side wall of the catheter body of the urinary catheter main body is provided with a catheter body sealing member in the circumferential direction. The catheter body sealing member corresponds to the position of the annular groove, so that the catheter body sealing member is attached to a urethral wall for sealing. At the same time, by forming the annular groove, the catheter body sealing member has a compression space, so that a persistent injury caused by the catheter body sealing member to the urethral wall may be avoided.

A specific structure of the urinary catheterization valve may be set to the structure of the urinary catheterization valve recorded in Embodiment 1 or Embodiment 2. The outer diameter of the urinary catheter main body is adapted to the size of urethra, and the size of the urinary catheterization valve may be set to be slightly greater than the size of the urethra. To further enhance slip resistance, an elliptic baffle fixing member may be disposed at the periphery of one end of the urinary catheter main body close to an external urinary control portion, to achieve engagement at the urethral orifice through the baffle fixing member, thereby further preventing slipping off.

As recorded in Embodiment 1 and Embodiment 2, in a possible implementation, the urinary catheterization valve may include a control valve body and a moving ball. One end of the control valve body is fixedly connected to an end portion of the urination end of the urinary catheter main body. The control valve body is internally provided with a spherical groove, a side wall of the control valve body extends outward to form a spherical cavity, and the spherical cavity is communicated with the spherical groove. The control valve body is provided with a urination channel communicated with the urinary catheterization channel in a penetrating manner in a length direction, and the urination channel penetrates through the spherical groove. The moving ball is movably disposed in the spherical groove. The spherical groove is in interference abutting with the moving ball, and the outer diameter of the urination channel is less than the outer diameter of the spherical groove. Further, the urinary catheter main body, the umbrella-shaped anti-off member, the baffle fixing member, the catheter body sealing member, and the control valve body are integrally formed and are all made of an elastic material conforming to biosecurity, which specifically may be silicone, to improve usage comfort while simplifying the overall structure. When a squeezing force is applied to an opposite side of the spherical cavity of the control valve body, the moving ball enters the spherical cavity from the spherical groove, so that the urination channel in the control valve body is opened, achieving the urination function. When urination is completed, the moving ball at the spherical cavity is squeezed to press the moving ball back to the spherical groove in the control valve body. The moving ball is attached to a side wall of the spherical groove to block and close the middle urination channel, achieving the urinary control effect.

In a possible implementation, by using an axial direction of the urinary catheter main body as a reference, there is a preset deflection angle between a straight line connecting a sphere center of the spherical cavity and a sphere center of the spherical groove and the axial direction of the urinary catheter main body, that is, the spherical cavity may be disposed at the position close to the baffle fixing member. The spherical cavity and the spherical groove may partially overlap as long as the urinary control and urination functions are not affected. The accommodation diameter of the spherical cavity may be slightly greater than the diameter of the moving ball, and the accommodation diameter of the spherical groove may be slightly less than the diameter of the moving ball, to achieve an interference fit with the moving ball, thereby improving the stability of urinary control.

The baffle fixing member may be disposed in an elliptic structure. A fixing hole is formed at a central part of the elliptic structure in a penetrating manner. The inner diameter of the fixing hole is adapted to the outer diameter of the urinary catheter main body. The elliptic structure is sleeved at the periphery of the end portion of the urination end of the urinary catheter main body through the fixing hole, to achieve engagement at the urethral orifice through the elliptic structure, thereby preventing the urinary catheter from contracting inward and slipping into the bladder. At the same time, disposing in the elliptic structure reduces the widths of both left and right sides of the baffle fixing member, facilitating concealment of the baffle fixing member in labia, so that the squeezing of both the left and right sides on the labia may be reduced.

When the end portion of the insertion end of the urinary catheter main body is disposed as the petal fixing member, the structure of the petal fixing member has been described in detail in Embodiment 1, which is disposed herein with reference to the structure in Embodiment 1. Correspondingly, an inner side wall of an end portion of the other end of a urinary catheterization portion may be provided with a plurality of strip-shaped grooves of a preset length at intervals in the axial direction. The petal fixing member includes a plurality of fan-shaped blades, and the plurality of fan-shaped blades are arranged at intervals, so that there is a gap between two adjacent fan-shaped blades, facilitating folding of the fan-shaped blades under external force. The petal fixing member is in a contracted state under external force with the diameter in the contracted state less than the diameter of the urethra, and is in an expanded state in a natural state with the diameter in the expanded state greater than the diameters of the urethra and the bladder orifice. The inner side wall of the insertion end of the urinary catheter main body is provided with the plurality of strip-shaped grooves of the preset length, facilitating contraction of the petal fixing member. The preset length is far less than the length of the urinary catheter main body.

At this time, the structural member and the urinary catheter main body may be compressed and loaded into a hand-tear sheath. Therefore, the catheter body sealing member 4 and the petal fixing member 2 of this disclosure are both required to be disposed as elastic structures, facilitating compression and loading into the hand-tear sheath. When in use, the hand-tear sheath is delivered to the bladder orifice via the urethral orifice, and a pushing rod is used to push the urinary catheter. At the same time, the hand-tear sheath is torn open and retracted; the petal fixing member 2 is released at the bladder orifice, and the position is appropriately adjusted; and the sheath continues to be retracted until the baffle is anchored at the urethral orifice. The position of the urinary catheter is fixed through the petal fixing member 2.

When the end portion of the insertion end of the urinary catheter main body is disposed as the umbrella-shaped anti-off member,

correspondingly, a traction guide wire may be used together, so that the urinary catheter may be directly delivered to the urethra from the urethral orifice of the patient through the traction guide wire while achieving snap fixing. A specific implementation process may refer to Embodiment 2.

The umbrella-shaped anti-off member may be formed by a plurality of arc-shaped umbrella segments that are connected in a surrounding manner in a circumferential direction. A surrounded region among the plurality of arc-shaped umbrella segments forms the guide wire abutting hollow cavity. Ends of the plurality of arc-shaped umbrella segments are connected to form the guide wire abutting end, and the other ends of the plurality of arc-shaped umbrella segments are connected to form the opening end of the guide wire abutting hollow cavity. The urine inlet is formed between every two arc-shaped umbrella segments, and the urine inlet and the guide wire abutting hollow cavity.

It should be noted that the structures, proportions, sizes, and the like shown in the accompanying drawings of the specification are only intended for matching the content disclosed herein and facilitating the understanding and reading of those skilled in the art rather than for limiting the implementable limiting conditions of this disclosure. Therefore, they have no technically substantial significance, and any structural modifications, changes in proportions, or adjustments in sizes, without affecting the effects and objectives that this disclosure can generate and achieve, shall still fall within the scope of the technical content disclosed in this disclosure. At the same time, the terms such as “upper”, “lower”, “left”, “right”, “middle”, and “one” as used in the specification are only for the convenience of description rather than for limiting the implementable scope of this disclosure, and changes or adjustments in their relative relationships, without substantially changing the technical content, shall also be deemed to fall within the implementable scope of this disclosure.

The above-described embodiments are only preferred embodiments of this disclosure and constitute no restriction in any form on this disclosure. Although this disclosure has been disclosed above with preferred embodiments, such preferred embodiments are not intended to limit this disclosure, and those skilled in the art can make some changes or modifications to equivalent embodiments with equivalent changes by reference to the technical content disclosed above without departing from the scope of the technical solutions of this disclosure. However, any simple revisions, equivalent changes, and modifications made to the above embodiments in accordance with the technical essence of this disclosure without departing from the content of the technical solutions of this disclosure shall still fall within the scope of the technical solutions of this disclosure.

What is claimed is:

1. A female urinary catheter, comprising:
a urinary catheter main body, wherein a urinary catheterization channel is formed in the urinary catheter main body in a penetrating manner, and an inner side wall of a catheter body of the urinary catheter main body is provided with a circle of an annular groove in a circumferential direction of the urinary catheter main body;
a petal fixing member, wherein the petal fixing member is fixed at an end portion of an insertion end of the urinary catheter main body in a surrounding manner in the circumferential direction of the urinary catheter main body;
a baffle fixing member, wherein the baffle fixing member is sleeved at an end portion of a urination end of the urinary catheter main body;
a catheter body sealing member, wherein the catheter body sealing member is disposed on an outer side wall of the catheter body of the urinary catheter main body and corresponds to a position of the annular groove; and
a urinary catheterization valve, wherein the urinary catheterization valve is fixedly disposed at the end portion of the urination end of the urinary catheter main body, and the baffle fixing member is located between the urinary catheterization valve and the urinary catheter main body, wherein
the catheter body sealing member and the petal fixing member are both made of an elastic material.

2. The female urinary catheter according to claim 1, wherein
a fixing hole is formed in a central part of the baffle fixing member in a penetrating manner, an inner diameter of the fixing hole is adapted to an outer diameter of the urinary catheter main body, and the baffle fixing member is sleeved at a periphery of the end portion of the urination end of the urinary catheter main body through the fixing hole;
the catheter body sealing member is of an annular protrusion structure, an outer diameter of the annular protrusion structure is greater than the outer diameter of the urinary catheter main body, and a size of the annular protrusion structure is adapted to an expansion size of female urethra; and
the petal fixing member comprises a plurality of fan-shaped blades, and the plurality of fan-shaped blades are arranged at the end portion of the insertion end of the urinary catheter main body at intervals in the circumferential direction of the urinary catheter main body.

3. The female urinary catheter according to claim 2, wherein
an inner side wall of the insertion end of the urinary catheter main body is provided with a plurality of strip-shaped grooves of a preset length, and the strip-shaped grooves are formed by extending from the end portion of the insertion end to the urination end for the preset length.

4. The female urinary catheter according to claim 3, wherein the urinary catheterization valve comprises:
an elastic valve body, wherein one end of the elastic valve body is fixedly connected to the end portion of the urination end of the urinary catheter main body, the elastic valve body is internally provided with a spherical hollow cavity, a side wall of the elastic valve body is provided with an inward hemispherical groove, the spherical hollow cavity is communicated with the hemispherical groove, the elastic valve body is further provided with an opening and closing channel communicated with the urinary catheterization channel in a penetrating manner in a length direction, and the opening and closing channel penetrates through the spherical hollow cavity;
a ball, wherein the ball is movably disposed in the spherical hollow cavity; and
an elastic sleeve, wherein the elastic sleeve is sleeved at a periphery of the elastic valve body, wherein
a size of the spherical hollow cavity is adapted to that of the ball, and an outer diameter of the opening and closing channel is less than an outer diameter of the spherical hollow cavity; the ball is made of a material conforming to biosecurity; and inner side walls of two ends of the elastic sleeve are provided with a circle of a limiting protrusion respectively in a circumferential direction of the elastic sleeve, two ends of an outer side wall of the elastic valve body are provided with a circle of a limiting groove respectively in a circumferential direction of the elastic valve body, and the limiting protrusion is snapped into the limiting groove.

5. The female urinary catheter according to claim 4, wherein
the urinary catheter main body, the petal fixing member, the baffle fixing member, the catheter body sealing member, and the elastic valve body are integrally formed and are all made of an elastic material conforming to biosecurity.

6. The female urinary catheter according to claim 3, wherein the urinary catheterization valve comprises:
a control valve body, wherein one end of the control valve body is fixedly connected to the end portion of the urination end of the urinary catheter main body, the control valve body is internally provided with a spherical groove, a side wall of the control valve body extends outward to form a spherical cavity, the spherical cavity is communicated with the spherical groove, the control valve body is further provided with a urination channel communicated with the urinary catheterization channel in a penetrating manner in a length direction, and the urination channel penetrates through the spherical hollow cavity; and
a moving ball, wherein the moving ball is movably disposed in the spherical groove, wherein
a size of the spherical groove is adapted to that of the moving ball, and an outer diameter of the urination channel is less than an outer diameter of the spherical groove.

7. A female urinary catheter, comprising:
a urinary catheter main body, wherein the urinary catheter main body is provided with a urinary catheterization channel in a penetrating manner in a length direction;
an umbrella-shaped anti-off member, wherein a top of the umbrella-shaped anti-off member is provided with a guide wire abutting end, the umbrella-shaped anti-off member is internally provided with a guide wire abutting hollow cavity, an opening end of the guide wire abutting hollow cavity is fixedly connected to an end portion of an insertion end of the urinary catheter main body, and a side wall of the umbrella-shaped anti-off member is provided with a urine inlet;
a baffle fixing member, wherein the baffle fixing member is sleeved at a periphery of a urination end of the urinary catheter main body;
a urinary catheterization valve, wherein the urinary catheterization valve is fixedly disposed at an end portion of the urination end of the urinary catheter main body, and the baffle fixing member is located between the urinary catheterization valve and the urinary catheter main body; and
a traction guide wire, wherein the traction guide wire and the urinary catheter main body are disposed independently, and one end of the traction guide wire abuts against the guide wire abutting end after sequentially passing through the urinary catheterization channel and the guide wire abutting hollow cavity, wherein
the umbrella-shaped anti-off member is made of an elastic material, and the guide wire abutting hollow cavity, the urinary catheterization channel, and the urine inlet are communicated with each other; and an outer diameter of the umbrella-shaped anti-off member in a natural state is greater than an outer diameter of the urinary catheter main body, and an outer diameter of the umbrella-shaped anti-off member in a deformed state is equivalent to the outer diameter of the urinary catheter main body.

8. The female urinary catheter according to claim 7, wherein
the baffle fixing member is of an elliptic structure, a fixing hole is formed in a central part of the elliptic structure in a penetrating manner, an inner diameter of the fixing hole is adapted to the outer diameter of the urinary catheter main body, and the elliptic structure is sleeved at a periphery of the end portion of the urination end of the urinary catheter main body through the fixing hole; and
an inner side wall of a catheter body of the urinary catheter main body is provided with a circle of an annular groove in a circumferential direction of the urinary catheter main body, an outer side wall of the catheter body of the urinary catheter main body is provided with a catheter body sealing member, the catheter body sealing member corresponds to a position of the annular groove, the catheter body sealing member is of an annular protrusion structure, an outer diameter of the annular protrusion structure is greater than the outer diameter of the urinary catheter main body, and a size of the annular protrusion structure is adapted to an expansion size of female urethra.

9. The female urinary catheter according to claim 8, wherein the urinary catheterization valve comprises:
a control valve body, wherein one end of the control valve body is fixedly connected to the end portion of the urination end of the urinary catheter main body, the control valve body is internally provided with a spherical groove, a side wall of the control valve body extends outward to form a spherical cavity, the spherical cavity is communicated with the spherical groove, the control valve body is provided with a urination channel communicated with the urinary catheterization channel in a penetrating manner in a length direction, and the urination channel penetrates through the spherical groove; and
a moving ball, wherein the moving ball is movably disposed in the spherical groove, wherein
the spherical groove is in interference abutting with the moving ball, and an outer diameter of the urination channel is less than an outer diameter of the spherical groove; and the urinary catheter main body, the umbrella-shaped anti-off member, the baffle fixing member, the catheter body sealing member, and the control valve body are integrally formed and are all made of an elastic material conforming to biosecurity.

10. A structural member for a female urinary catheter, wherein the female urinary catheter comprises a urinary catheter main body, and the urinary catheter main body is provided with a urinary catheterization channel; the structural member comprises: a urinary catheterization valve, wherein the urinary catheterization valve is disposed at a urination end of the urinary catheter main body; and the structural member further comprises:
a petal fixing member, wherein the petal fixing member is disposed at an insertion end of the urinary catheter main body in a surrounding manner in a circumferential direction of the urinary catheter main body;
or
an umbrella-shaped anti-off member, wherein the umbrella-shaped anti-off member is provided with a guide wire abutting end, the umbrella-shaped anti-off member is internally provided with a guide wire abutting hollow cavity, an opening end of the guide wire abutting hollow cavity is fixedly connected to the insertion end of the urinary catheter main body, the umbrella-shaped anti-off member is provided with a urine inlet, and the guide wire abutting hollow cavity, the urinary catheterization channel, and the urine inlet are communicated with each other.
